(19) United States
(12) Patent Application Publication
(10) Pub. No.: US 2013/0280777 A1
(43) Pub. Date: Oct. 24, 2013

(54) DIGESTER

(71) Applicant: Christopher Muir, Devon (GB)

(72) Inventor: Christopher Muir, Devon (GB)

(73) Assignee: ch4e Limited, Devon (GB)

(21) Appl. No.: 13/820,874

(22) PCT Filed: Sep. 5, 2011
(86) PCT No.: PCT/GB2011/001302

§ 371 Date: May 14, 2013

(30) Foreign Application Priority Data
Sep. 6, 2010 (GB) 1014693.4

(51) Int. Cl. C12M 1/107

(52) U.S. Cl. CPC C12M 21/04
USPC 435/167; 435/290.4

(57) ABSTRACT

The present invention relates to a digester (10) for a digester plant (1), comprising a digester tank (11) defining a digester tank chamber (62) and having a module opening (69) and a digester module (72) located in the module opening and extending into the digester tank chamber (62). The digester module is removably attached to the digester tank and comprises a heater (84) for heating material in the digester tank (11) together with at least one pipe (94; 96; 118) with an inlet (98; 108) and an outlet (102 104; 110; 122), one of which is inside the digester tank chamber 62 and the other of which is outside the digester tank chamber (62). Said at least one pipe may be: a supply pipe (94) for supplying material to the digester tank, the supply pipe having an inlet (98) outside the tank chamber and an outlet (102, 104) inside the tank chamber; a withdrawal pipe 96 for withdrawing material from the digester tank, the withdrawal pipe having an inlet 108 inside the tank chamber and an outlet (110) outside the tank chamber; or a gas withdrawal pipe (118) for withdrawing gas from the digester tank, the gas withdrawal pipe having an inlet inside the tank chamber and an outlet (122) outside the tank chamber.

The present invention relates to a digester, in particular, a digester for anaerobic digestion. The present invention also relates to a digester plant and methods for digesting biodegradeable feedstocks.

The financial cost associated with generating energy from conventional coal and oil based resources has increased significantly in recent years and shows no sign of abating. The harm caused to the environment by the extraction and subsequent use of these resources to generate power is now receiving attention from governments and other regulatory bodies around the world. As a result, there is currently a keen interest in identifying and exploiting alternative sources of energy. The increased appreciation of the importance of environmental issues has also lead to a host of new regulations being introduced to monitor and control the manner in which waste and potentially harmful substances are managed and disposed of. These different factors combine to create an environment which is particularly testing for small-to-medium scale operations, such as farms, small housing estates, holiday parks and campsites, and even temporary military instillations, which cannot benefit from the efficiencies in terms of energy usage and waste management available to larger scale operations. There is therefore a need to develop systems to help small-to-medium scale operations meet their energy needs and environmental obligations.

Anaerobic digestion, otherwise known as anaerobic fermentation, is a process that can be used to convert biodegradable material into biogas and a digestate composed of a liquid fraction and a solid fraction. A wide range of biodegradeable materials can be used as the feedstock for the digestion process such as farmyard slurry, domestic food and garden waste, human effluent, catering and food processing waste, waste from abattoirs and mechanically-separated municipal waste (i.e. waste in which the non-biodegradeables have been removed). The treated waste or digestate can be used as fertiliser in which the liquid and solid fractions are retained together, or they can be separated with the liquid fraction used as a liquid fertiliser and the solid fraction composted before being used as a fertiliser. Biogas is a mixture of methane gas and primarily carbon dioxide gas. The methane gas can be used as a biofuel in vehicles and the like, or it can be used to generate electricity or heat. Electricity can be used directly by the digester operator, by others in the surrounding area or it can be supplied to the national power grid to generate financial revenue. Heat can be used directly or used to heat water again either by the digester operator or by others.

Advantageously, heat generated can be recycled back to the digester to support the digestion of further biodegradeable material.

Digester plants are known in which biodegradable waste is fed to a number of digester tanks. The waste is heated in the tanks and the biogas is drawn off the top of the tanks. During installation of the digester plant, heaters, pumps, waste inlets and outlets, and gas outlets must be installed in the tanks. This can be a time consuming process that is usually carried out on site and it is necessary to work inside the confined space of the tank. Further, if the digestion plant requires maintenance or repair then the digester tanks must be drained and a worker may be required to carry out work inside the tank. This is highly undesirable as the conditions within a digester tank are unpleasant and can present a significant health and safety risk to the worker. There therefore currently exists a need to address one of more of the problems outlined above in relation to existing digesters and digester plants.

An object of the present invention is to obviate or mitigate one or more of the problems outlined above in relation to efficient and environmentally acceptable energy generation.

Another object of the present invention is to provide an improved digester or digester plant. It is desirable that the improvement contributes to enabling the digester or digester plant to be manufactured, installed, maintained and/or repaired more easily and/or safely than existing digesters and digester plants.

According to a first aspect of the present invention there is provided a digester for a digester plant, comprising:
a digester tank defining a digester tank chamber and having a module opening;
a digester module located in the module opening and extending into the digester tank chamber, wherein the digester module is removably attached to the digester tank and comprises a heater for heating material in the digester tank and at least one pipe having an inlet and an outlet, one of said inlet and outlet being inside the tank chamber and the other of said inlet and outlet being outside the tank chamber.

Said at least one pipe is selected from the group consisting of:
a supply pipe for supplying material to the digester tank, the supply pipe inlet being outside the tank chamber and the supply pip outlet being inside the tank chamber;
a withdrawal pipe for withdrawing material from the digester tank, the withdrawal pipe inlet being inside the tank chamber and the withdrawal pipe outlet being outside the tank chamber; and
a as withdrawal pipe for withdrawing gas from the digester tank, the gas withdrawal pipe inlet being inside the tank chamber and the gas withdrawal pipe outlet being outside the tank chamber.

The present invention may thus provide a digester for a digester plant, comprising:
a digester tank defining a digester tank chamber and having a module opening;
a digester module located in the module opening and extending into the digester tank chamber, wherein the digester module is removably attached to the digester tank and comprises a heater for heating material in the digester tank and at least one of the following:
a supply pipe for supplying material to the digester tank, the supply pipe having an inlet outside the tank chamber and an outlet inside the tank chamber;
a withdrawal pipe for withdrawing material from the digester tank, the withdrawal pipe having an inlet inside the tank chamber and an outlet outside the tank chamber;
a gas withdrawal pipe for withdrawing gas from the digester tank, the gas withdrawal pipe having an inlet inside the tank chamber and an outlet outside the tank chamber.

In this way, key components of the digester, such as the heater and one or more of the supply pipe, withdrawal pipe and gas withdrawal pipe can be safely and securely located in the optimum position within the digester tank. This provides a relatively straightforward means of ensuring that these key components are appropriately arranged to ensure the digester works correctly, while also simplifying the installation, maintenance and repair of these components. It also enables the manufacturer of the digester to securely lock the digester module in place within the opening in the digester tank before it is used, thereby preventing the operator of the digester or anyone else from gaining unauthorised access into the tank, which might otherwise lead to key components being damaged or harm to the person entering the tank.

It is particularly preferred that the digester is an anaerobic digester, or is configured to operate as an anaerobic digester. That is, it is preferred that the digester is operated with the digester tank chamber containing no air, or substantially no air. Preferably the digester tank is sealable so as to prevent the egress of air into the tank during use. In this way, anaerobic conditions with the digester tank chamber can be maintained during use to ensure efficient conversion of the biodegradeable feedstock to biogas and digestate.

It is preferred that the digester module comprises at least two pipes, each with an inlet inside or outside the digester tank and an outlet inside or outside the digester tank. Preferably the digester module comprises at least two of the supply pipe, the withdrawal pipe and the gas withdrawal pipe. In a preferred embodiment the digester module comprises the supply pipe, the withdrawal pipe and the gas withdrawal pipe.

The digester tank may be of any appropriate size, shape and volume to accommodate the quantity and type of biodegradeable feedstock available and the desired output of biogas and/or digestate. While the digester tank may comprise a single digester module opening to receive a single digester module, the digester tank may comprise two or more module openings, each module opening having a dedicated digester module located therein. Alternatively, the digester tank may comprise a single module opening configured to receive two or more digester modules. As a further alternative, the digester tank may comprise two or more module openings, each of which is independently arranged to receive one or more digester modules.

The outlet of the supply pipe may be towards the top of the tank chamber. The supply pipe may comprise at least two outlets located inside the tank chamber. The withdrawal pipe may extend towards the bottom of the tank chamber and the inlet may be towards the bottom of the tank chamber. The inlet of the gas withdrawal pipe may be towards the top of the tank chamber. It is preferred that the heater comprises a length of tube having a water inlet and a water outlet outside the tank chamber. The length of tube is preferably coiled.

The digester tank may comprise a first flange and the digester module may comprise a second flange, the second flange being detachably attached to the first flange. It is preferred that the two flanges are secured together by the manufacturer of the digester either off-site or on-site but before the digester operator is left to use the digester. Some form of tamper-evident seal may be used so that the manufacturer or service engineering will know in future whether the operator or other person as gained or tried to gain unauthorised access into the digester tank chamber by unsecuring the two flanges. The supply pipe inlet and/or the withdrawal pipe outlet and/or the gas withdrawal pipe outlet is preferably attached to the first flange. The digester module preferably comprises a cage for protecting the supply pipe and/or the withdrawal pipe and/or the gas withdrawal pipe. Any suitable cage may be used but it preferably comprises a mesh. The cage preferably incorporates or encompasses the coiled tube of the heater.

The supply pipe is preferably operatively connected to a material dosing tank, which may incorporate a controllable valve to enable the amount and flow rate of biodegradeable feedstock passing from the dosing tank to the digester tank to be regulated and adjusted as necessary. It is preferred that the supply pipe is operatively connected to a hydrolysis tank configured to mix the biodegradeable feedstock with water before being fed to the digester tank. The hydrolysis tank is ideally operatively connected to the supply pipe via a filter or separator arranged to remove solids and any other undesirable substances from the feedstock before it is passed to the digester tank. It is particularly preferred that the dosing tank is provided in between the hydrolysis tank (and its filter if present) and the digester tank. That is, it is preferred that the feedstock is first hydrolysed, then filtered and then passed to the dosing tank from where it is fed to the digester tank.

Preferably the gas withdrawal pipe is operatively connected to a gas storage vessel. The gas storage vessel may have one or more outlets operatively connected to a single type of end use application, e.g. a single CHP generator set, or two or more types of end use application, e.g. a CHP generator set and a long term gas storage vessel.

A second aspect of the present invention provides a module for use with the digester according to the above defined first aspect and preferred embodiments of the present invention.

A third aspect of the present invention provides a digester plant comprising at least one digester in accordance with the digester according to the above defined first aspect and preferred embodiments of the present invention.

It is preferred that the digester plant according to the third aspect of the present invention comprises a plurality of digesters, one, more than one, or all of which are in accordance with the first aspect of the present invention.

Two or more of the plurality of digesters may be connected to a controller or arranged to operate simultaneously, sequentially or with a lag between digestion starting in one digester and then being started in another digester. Using two or more digesters sequentially or with deferred starting times (i.e. with a lag) enables a more constant supply of biogas to be generated. The digesters can be operated so as to maximise biogas generation but may be more appropriate to aim to generate a supply of biogas which can be maintained at a substantially constant level below the maximum, rather than risking fluctuations in biogas flow rate which might occur if operating parameters are stretched to their limits to produce as much biogas as possible. It will be appreciated that variations in biogas generation can be reduced by using a larger number of digesters running sequentially or with a lag. While any desirable number of digesters can be used, it is currently envisaged that for small-to-medium sized applications 2 to 10 digesters may be adequate.

The digester tank chamber may have any desirable capacity and may be operated at any appropriate temperature using whatever flow rate of biodegradeable feedstock is available and sustainable, but it is envisaged that the present invention is particularly suitable for use in small-to-medium sized applications where the quantity and flow rate of biogas produced is sufficient to power 50 to 200 kWh generator sets.

By way of a first example, the digester tank may have an optimum working capacity for biodegradeable feedstock of 100 tonnes and incorporate two module openings, each receiving a single digester module incorporating a heater, supply pipe, withdrawal pipe and gas withdrawal pipe. Based on a retention time for the feedstock within the digester tank of around 20 days, this digester tank could be controlled to generate biogas at a substantially constant flow rate of around 5 to 7 m³ per hour. Four such tanks could be used simultaneously to digest up to around 8000 tonnes of feedstock material per year and to produce biogas at a substantially constant rate of around 20 to 28 m³ per hour, which the skilled person will appreciate is sufficient to drive a 50 kWh generator set.

Calculations have demonstrated that using six such digester tanks operating under similar conditions should enable around 12000 tonnes of feedstock material to be processed per year to drive a 75 kWh generator set. While further calculations suggest that it should be possible to drive a 100 kWh generator set using eight digesters digesting 16000 tonnes of feedstock.

The digester plant preferably comprises a feedstock hydrolysis tank that is operatively connected to the or each digester. The hydrolysis tank is configured to mix the biodegradeable feedstock with water before the feedstock is sent to the digester(s). The hydrolysis tank is preferably configured to be able to hydrolyse the feedstock while generating essentially no gaseous products. A filter or separator is preferably provided in between the hydrolysis tank and the digester(s) to ensure solids and any other undesirable substances are removed from the hydrolysed feedstock before it is passed to the digester(s).

The digester plant preferably comprises a feedstock dosing tank to temporarily store and then controllably feed the feedstock to the digester(s).

It is particularly preferred that the digester plant comprises both a dosing tank and hydrolysis tank with a filter, with the dosing tank being located in between the hydrolysis tank and the digester tank. In this way, the feedstock is first hydrolysed, then filtered and then passed to the dosing tank from where it can be fed to the digester(s) as required to maintain digestion such that a constant supply of biogas is generated.

The digester plant preferably comprises at least one biogas store operatively connected to the digester(s) to receive and store biogas generated during the digestion process. The digestion plant may incorporate one or more short term biogas storage vessels in which biogas is held only temporarily before being passed elsewhere, e.g. to a generator set. Alternatively or additionally, the digester plant may incorporate one or more long term biogas storage vessels in which biogas can be stored for weeks, months or longer for use at a later date.

The digester plant preferably comprises at least one generator operatively connected to the digester(s) to generate power from the biogas produced in the digester(s). Any appropriate type of generator may be used, such as a combined heat and power (CHP) generator. The generator may be at least a 25 kWh generator set, more preferably at least a 50 kWh generator set. The generator may be at least a 75 kWh generator set and may be at least a 100 kWh generator set or larger. Preferably the generator is a 25 to 150 kWh generator set, more preferably the generator is a 50 to 100 kWh generator set.

A fourth aspect of the present invention provides a method for digesting a biodegradeable material using a digester plant comprising one or more digesters, at least one of said digesters comprising:
a digester tank defining a digester tank chamber and having a module opening;
a digester module located in the module opening and extending into the digester tank chamber, wherein the digester module is removably attached to the digester tank and comprises a heater for heating biodegradeable material in the digester tank and at least one pipe having an inlet and an outlet, one of said inlet and outlet being inside the tank chamber and the other of said inlet and outlet being outside the tank chamber,
wherein the method comprises:
providing biodegradeable material in the digester tank chamber;
exposing the biodegradeable material in the digester tank chamber to bacteria capable of digesting the biodegradeable material to produce biogas;
operating the heater to heat the biodegradeable material; and
removing biogas produced by digestion of the biodegradeable material by the bacteria.

The digester plant preferably comprises a plurality of digesters and the method preferably involves carrying out the method set out above within two or more of said plurality of digesters simultaneously, sequentially or with a lag between the time at which biodegradeable material in each digester tank is exposed to the bacteria.

When the multiple digesters are operated simultaneously the biodegradeable material in each digester tank is exposed to the bacteria at the same time, or substantially the same time.

When the multiple digesters are operated sequentially the biodegradeable material in a first of the digester tanks is exposed to the bacteria at a first time and then the biodegradeable material in a second of the digester tanks is exposed to the bacteria at a second time, the second time being after the majority or substantially all of the biogas has been removed from the first of the digester tanks.

When the multiple digesters are operated with a lag the biodegradeable material in a first of the digester tanks is exposed to the bacteria at a first time and then the biodegradeable material in a second of the digester tanks is exposed to the bacteria at a second time, the second time being after the first time but before the majority or substantially all of the biogas has been removed from the first of the digester tanks. The second time is preferably later than the first time but occurs while the biodegradeable material in the first of the digesters is being exposed to bacteria.

It is preferred that the method is effected under anaerobic (i.e. no oxygen) conditions such that the method is an anaerobic digestion process. The method is preferably carried out under conditions which produce biogas at a substantially constant flow rate, which may be less than the maximum possible flow rate. The method is preferably effected in the or each digester to produce biogas at a rate of at least around 2 m³ per hour, more preferably at least around 3, 4, or 5 m³ per hour. For the small-to-medium scale applications for which the present invention is especially amenable it is preferred that the method is carried out to produce biogas at a flow rate of around 2 to 10 m³ per hour, more preferably around 4 to 8 m³ per hour, and most preferably around 5 to 7 m³ per hour.

The method is preferably carried out under mesophilic conditions. That is, mesophilic bacteria are preferably employed with the biodegradeable material in the digester tank heated to a temperature of around 35 to 42° C., more preferably around 36 to 38° C. While the method can be carried out under thermophilic conditions at 52 to 55° C. using thermophilic bacteria, since they are more sensitive to fluctuations in operating conditions they are less robust than mesophlilic bacteria with the result that the digestion process may be less reliable and produce a less constant supply of biogas over extended periods of time.

The biodegradeable material is preferably exposed to bacteria in the digester tank chamber for up to around 50 days, more preferably up to around 25 days and still more preferably up to around 20 days. The time period over which the biodegradeable material is exposed to the bacteria is preferably at least around 8 to 10 days, more preferably at least around 12 to 18 days. It is particularly preferred that the biodegradeable material is exposed to the bacteria for around 10 to 20 days, more preferably around 12 to 18 days. It will be appreciated that the shorter the exposure time (which may be considered a ‘retention time’ for the biodegradeable material within the digester tank chamber), the greater the number of times the method can be performed in a finite period of time, e.g. per month or per year.

One approach to reducing exposure or retention time is to carry out pre-digestion hydrolysis of the biodegradeable material. That is, it is preferred that the method comprises hydrolysis of the biodegradeable material before it is provided in the digester. Hydrolysis should be effected under suitable conditions over a sufficient period of time to hydrolyse the biodegradeable material to the extent that the time over which the biodegradeable material needs to be exposed to the bacteria is reduced, preferably significantly reduced, by at least 5 to 10%, and preferably longer, such as 15 to 20% or more as compared to the time exposure time that would be required if no pre-digestion hydrolysis has been carried out. Pre-digestion hydrolysis may be carried out at around 30 to 80° C., more preferably around 40 to 70° C., and most preferably around 60° C. Hydrolysis may be carried out for up to around 7 days, more preferably around 1 to 6 days, and most preferably around 3 days.

The method preferably further comprises filtering the hydrolysed biodegradeable material before providing it in the digester tank chamber. Filtration is preferably carried out to remove solids and any other species that it would be undesirable to pass into the digester tank chamber.

It is preferred that the method comprises initially providing the biodegradeable material in a dosing tank from which it is controllably provided in the digester tank chamber.

In a particularly preferred embodiment, the biodegradeable material is hydrolysed and then fed to the dosing tank via a filter before being provided in the digester tank chamber.

The method preferably further comprises feeding the biogas removed from the digester tank chamber to a biogas store.

The removed biogas is preferably fed to a generator, of any appropriate size and power rating.

The generator is preferably operated to generate heat. A portion of the generated heat is preferably returned to the digester tank chamber to heat further biodegradeable material in the digester tank chamber. The portion of heat returned may be at least 10 to 20%, more preferably 20 to 80%, still more preferably around 40 to 60%, and is most preferably around 50% of the heat generated by the generator.

The invention may comprise any combination of the features and/or limitations referred to herein, except combinations of such features as are mutually exclusive. In particular, the module may comprise all four of the supply pipe, the withdrawal pipe, the heater and the gas withdrawal pipe.

Embodiments of the present invention will now be described, by way of example only, with reference to the accompanying drawings, in which:

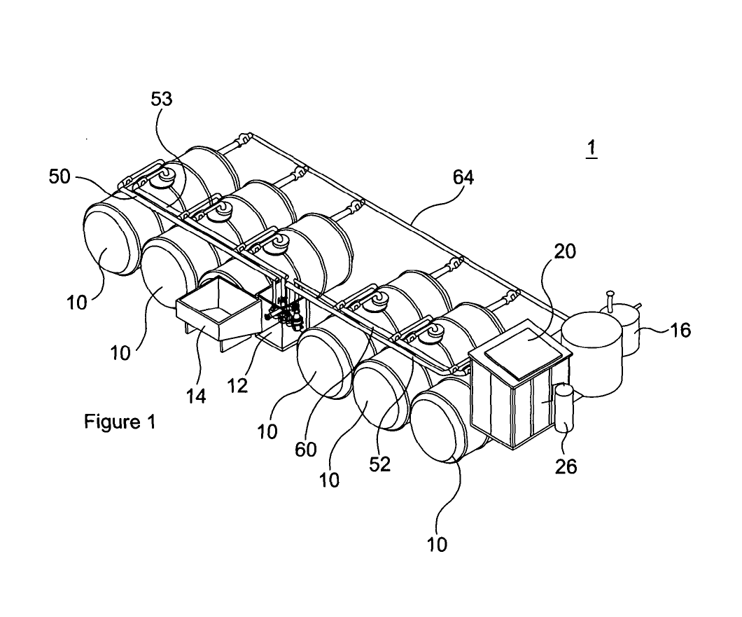

FIG. 1 schematically shows a digester plant;

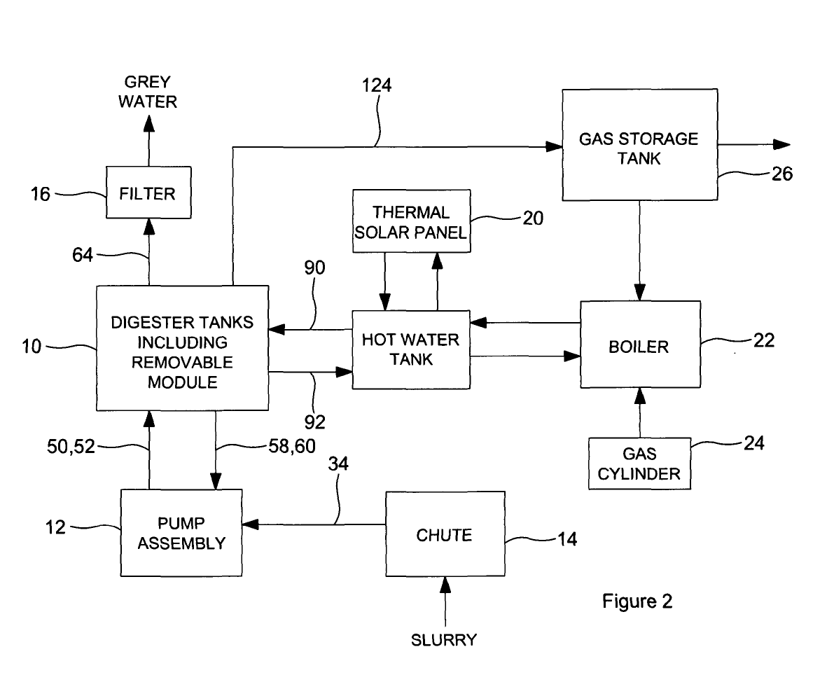

FIG. 2 shows a box diagram of the digester plant of FIG. 1;

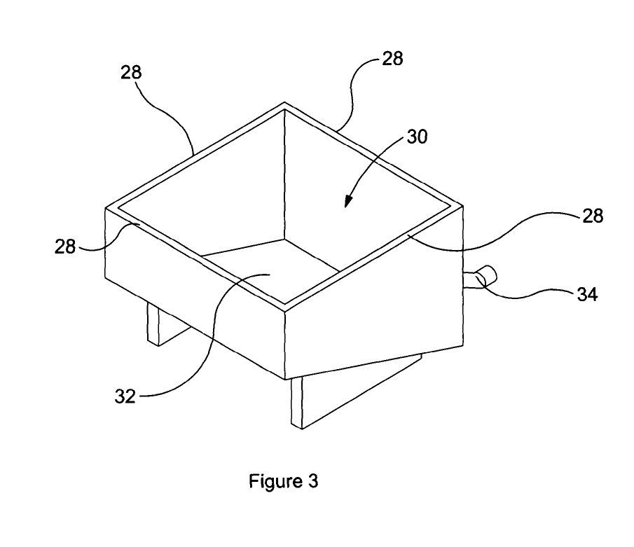

FIG. 3 schematically shows the chute of FIG. 1;

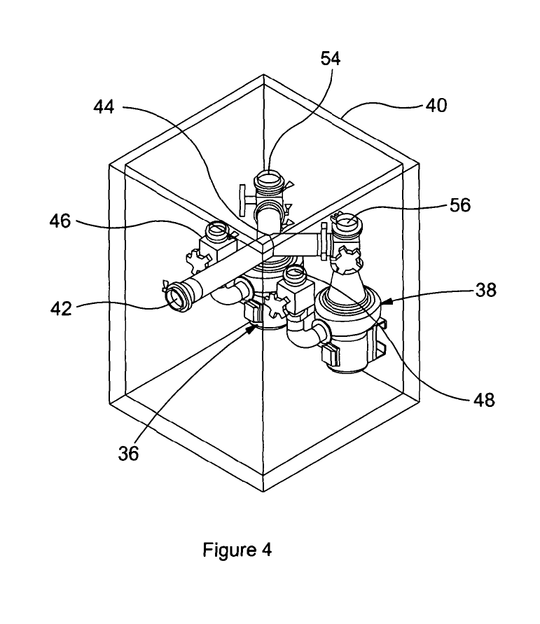

FIG. 4 schematically shows the pump assembly of FIG. 1;

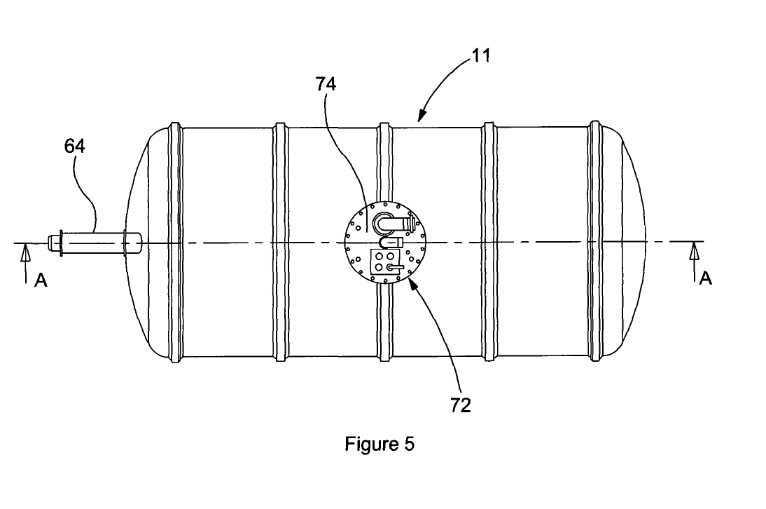

FIG. 5 schematically shows the digester of FIG. 1;

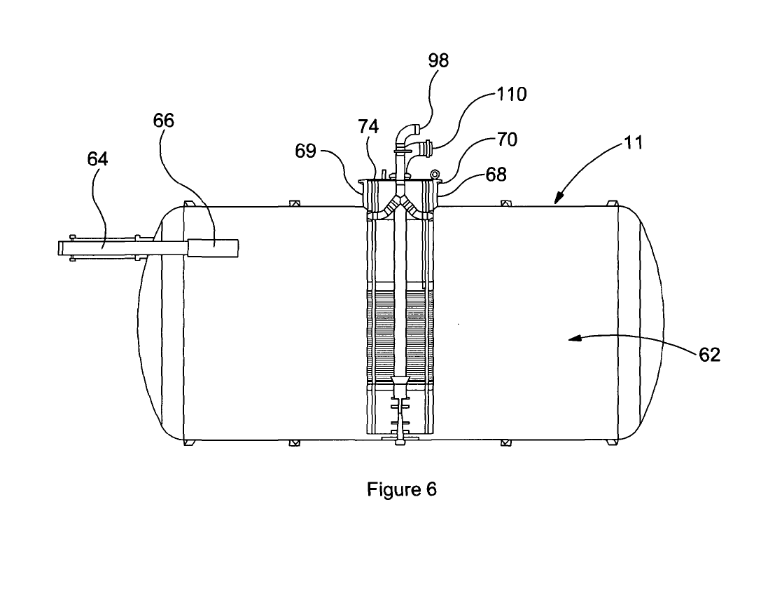

FIG. 6 schematically shows the view A-A of FIG. 5;

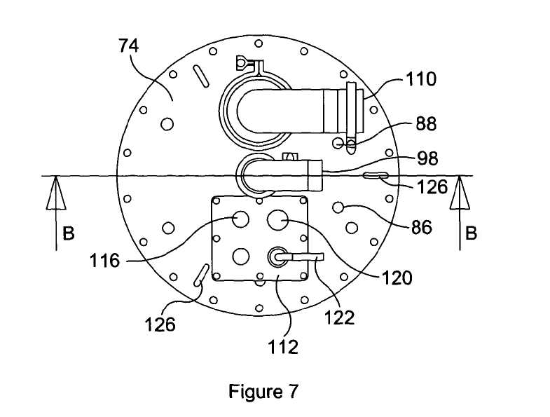

FIG. 7 schematically shows a top view of the digester module of FIG. 6;

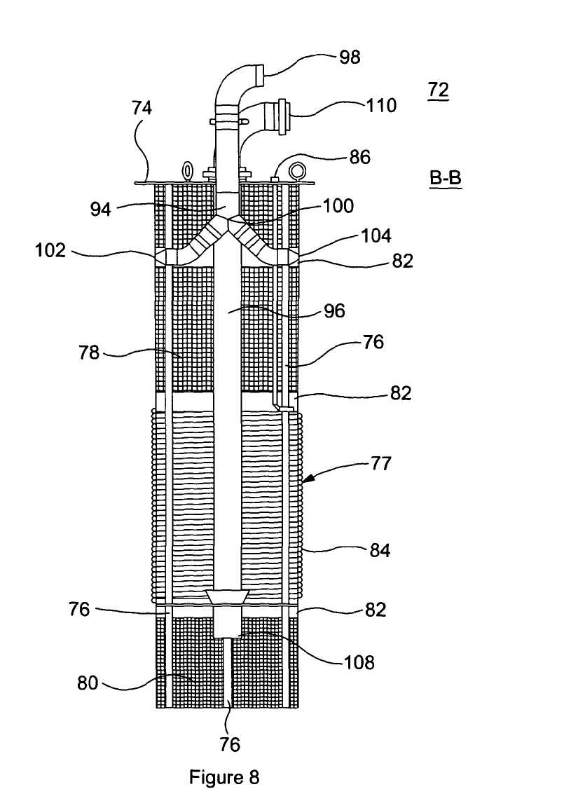

FIG. 8 schematically shows the view B-B of FIG. 7; and

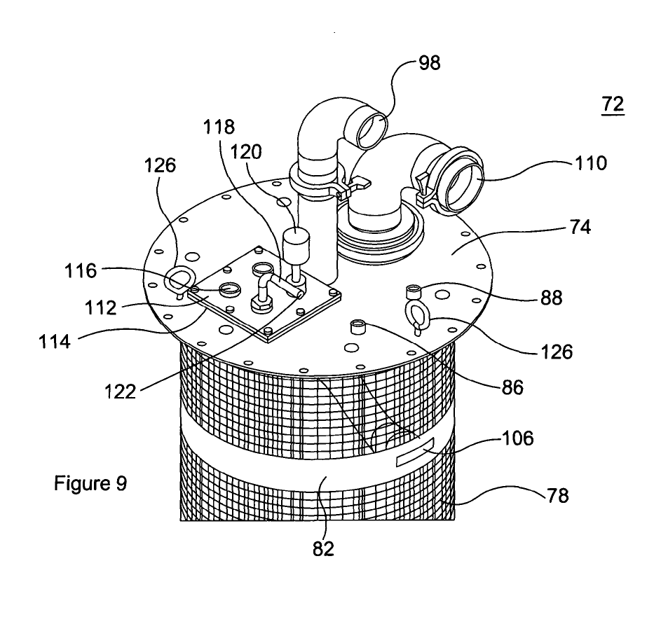

FIG. 9 schematically shows an enlarged view of the top of the digester module of FIGS. 7 and 8.

FIGS. 1 and 2 show an anaerobic digester plant 1. The plant 1 comprises six digesters 10, a pump assembly 12 and a chute 14 for slurry or the like. The chute 14 is connected to the pump assembly 12 which takes slurry fed into the chute 14 and feeds it to the digesters 10. The pump assembly 12 also re-circulates slurry already in the digesters 10. The digesters 10 are also provided with an outlet which is attached to a filter 16. This allows grey water to be drained from the tanks if necessary. The plant 1 may further comprise a pre-digestion hydrolysis tank (not shown), which receives slurry before it is fed to the digesters 10. The hydrolysis tank is operated so as to combine the untreated slurry with water, optionally in the presence of heating, so as to effect partial or substantially complete hydrolysis of the slurry before it is sent to the digesters 10. In this way, the time period over which the slurry needs to reside within the digesters 10 can be reduced, thereby increasing the amount of slurry that can be processed over a finite period of time.

Each digester 10 includes a heater in the form of a heater coil (not shown in FIG. 1 or 2). The heater coil is supplied with hot water from a hot water tank 18. The water in the hot water tank 18 is heated by either a thermal solar panel 20, a conventional boiler 22, a combination thereof, or by any other suitable method. The heater coil heats the slurry in the digesters 10 resulting in anaerobic fermentation (otherwise known as anaerobic digestion) of the slurry which produces methane (CH₄).

This methane gas is drawn off the digesters 10 and fed to a gas storage tank 24. The methane gas stored in the storage tank 24 can be used to power the boiler 22. If necessary, a propane gas cylinder 26 may be provided for powering the boiler 22 during start-up.

The methane gas generated by the anaerobic digester plant 1 can be used for a variety of purposes. It may be used to power a generator or used to power a boiler to supply hot water to buildings, for example. The treated slurry, known as digestate, can be used as fertiliser.

FIG. 3 shows the slurry chute 14 which comprises four side walls 28, defining a chute opening 30, and an inclined base 32. A chute pipe 34 is connected to the chute and at a first end opens into the lowest point of the chute. A second end of the chute pipe 34 is connected to the pump assembly 12.

FIG. 4 shows the pump assembly 12 which comprises first and second chopper pumps 36, 38 located within a pump assembly housing 40. A pump inlet pipe 42 is provided which is connected to the outlet (or second end) of the chute pipe 34. A Y-piece 44 is attached to the pump inlet pipe 42 and allows slurry to be drawn into the first and second chopper pumps 36, 38. The first and second pumps 36, 38 are provided with first and second pump outlets 46, 48 respectively. The first and second pump outlets 46, 48 are connected to first and second tank supply lines 50, 52, each line 50, 52 being arranged to supply slurry to three individual digesters 10. The first and second pumps 36, 38 are also provided with first and second pump inlets 54, 56 respectively. The first and second pump inlets 54, 56 are connected to first and second tank withdrawal lines 58, 60, each line 58, 60 arranged to withdraw slurry from three individual digesters 10.

FIGS. 5 and 6 show a digester 10. The digester 10 comprises a digester tank 11 and a removable digester module 72. The digester tank 11 is made from fibre-glass and defines a digester chamber 62 having a volume of approximately 30,000 litres. As will be readily apparent to one skilled in the art, the digester chamber 62 may have any other suitable volume. A grey water outlet pipe 64 is in fluid communication with the chamber 62 and is provided with a filter 66 at a first end which is inside the chamber 62. The second end of the grey water outlet pipe 64 is attached to the plant filter 16. The grey water outlet pipe 64 is arranged to drain grey water from the digester tank 11. The digester tank 11 has a cylindrical wall 68 extending from an upper surface of the tank 11 which defines a module opening 69 that leads to the chamber 62. An annular flange 70 is attached to the upper edge of the cylindrical wall 68. The digester module 72 is located in the opening and extends to the bottom of the digester tank 11. The digester module 72 includes a flange plate 74, having a plurality of circumferentially arranged holes, and is bolted to the flange 70 of the digester tank 11 through these holes in order to secure the module 72 to the tank 11. Referring now to FIGS. 7, 8 and 9 the digester module 72 comprises a substantially circular flange plate 74 having a plurality of elongate chassis members 76 circumferentially arranged and extending in the general axial direction of the module. A first cylindrical portion of mesh 78 is wrapped around an upper portion of the elongate chassis members 76. The first mesh 78 is adjacent to the flange plate 74 and extends over a portion of the length of the chassis members 76. A second cylindrical portion of mesh 80 is wrapped around a lower portion of the elongate chassis members 76. The second mesh 80 extends over a portion of the length of the chassis members 76 and is spaced from the first mesh 78. In this embodiment the second mesh 80 is finer than the first mesh 78. The first and second meshes 78, 80 are secured to the elongate chassis members 76 by annular metal bands 82 provided over the mesh 78, 80.

The digester module 72 is provided with a heater coil 84 in the region between the first mesh 78 and the second mesh 80. The heater coil 84 is in the form of a metal tube wrapped around the elongate chassis members 76. The heater coil 84 has a fluid inlet 86 and a fluid outlet 88 that are located on the flange plate 74. The fluid inlet 86 and fluid outlet 88 are arranged to be connected to a water supply line 90 and a water return line 92 that are in fluid communication with the hot water tank 18. During use, a continuous flow of hot water is supplied from the hot water tank 18 to the heater coil 84 through water supply line 90 and is continuously withdrawn from the heater coil 84 and returned to the hot water tank 18 through the water return line 92.

The digester module 72 further comprises a module supply pipe 94 and a module withdrawal pipe 96, The module supply pipe 94 is attached to and extends through the flange plate 74 and comprises an inlet 98 and a Y-piece 100 having first and second outlets 102, 104. The ends of the Y-piece are secured to an upper annular band 82 which has two openings 106 that are aligned with the first and second outlets 102, 104. The module withdrawal pipe 96 is attached to and extends through the flange plate 74 and comprises an inlet 108 and an outlet 110. The module withdrawal pipe 96 extends along the majority of the length of the digester module 72 and is positioned inside the first and second meshes 78, 80 and the heater coil 84. The inlet 108 is positioned towards the bottom of the digester module 72 in the vicinity of the second mesh 80. The inlet 98 of the module supply pipe 94 and the outlet 110 of the module withdrawal pipe 96 are arranged to be connected to a tank supply line 50, 52 and a tank withdrawal line 58, 60 respectively. During use, the pump assembly continuously re-circulates slurry by withdrawing it from the associated digesters 10 through a tank withdrawal line 58, 60 and supplying it back to the digester 10 through a tank supply line 50, 52.

The first and second meshes 78, 80 and the heater coil 84 act as a cage 77 to protect the supply pipe 94, withdrawal pipe 96 and gas withdrawal pipe 118. The cage 77 helps to protect the aforementioned components from damage during transportation of the digester module 72, installation of the digester module 72 and during use of the digester module 72.

Referring specifically to FIG. 9, the flange plate 74 is provided with a cover plate 112 that seals an access opening in the flange plate 74. The cover plate 112 is bolted to the flange plate 74 and a gasket seal 114 is disposed between the two. The cover plate 112 has four access holes 116 with a gas withdrawal pipe 118 extending through a first hole and a dip stick 120 extending through a second hole. In this particular embodiment the third and fourth holes are sealed with plugs (not shown) but the plugs can be removed if it is necessary to insert a device through the respective hole. The gas withdrawal pipe 118 has an inlet (not shown) which, when the module is installed, is located inside the digester tank 11 and is positioned towards the top of the module 72, and an outlet 122 located outside the digester tank 11. The gas outlet 122 is arranged to be attached to a main gas line 124 which is in fluid communication with the gas storage tank 26.

During use, the gas withdrawal pipe 118 continuously withdraws methane gas from the top of the digester tank 11 and feeds it to the gas storage tank 26. The dip stick 120 extends to the bottom of the module 72 and can be used to take samples from inside the tank 11. The flange plate 74 is further provided with two rings 126 which can be used to lift the digester module 72 using machinery such as a crane or tractor, for example.

The digester tank 11 is typically a fibre glass tank which can be manufactured entirely separately from the digester module 72. This improves the ease of manufacture and reduces the overall cost. The components of the digester module 72 such as the flange 74, chassis members 76, mesh 78, 80, annular bands 82, heater coil 84, module supply pipe 94, module withdrawal pipe 96 and gas withdrawal pipe 118 are made from 316 stainless steel.

This particular material is hard-wearing and corrosion resistant which is important since the environment within the digester 10 is particularly harsh and includes many corrosive chemicals.

The digester plant 1 shown in FIG. 1 may be installed as follows. A pit is dug and the six digester tanks 11 are located within the pit with the flange 70 of each tank 11 substantially at ground level. The pump assembly 12 is also located in the pit. For embodiments including a pre-digestion hydrolysis tank (not shown) this may also be provided in the pit or may be provided at a separate location. A hole is drilled in an end of each digester tank 11 and a grey water pipe 64 and filter 66 is installed in each tank. The grey water pipes 64 of each tank 11 are then connected together and connected to the filter 16. For some installations it is not necessary to have a grey water pipe 64 and associated filter 16. A digester module 72 is located into each digester tank 11 and the flange plate 74 of the module 72 is bolted to the flange of the tank 11. The supply inlet 98 of the digester modules 72 of three digesters 10 are then connected to the first pump outlet 46 of the first pump 36 using the first tank supply line 50. Similarly, the supply inlet 98 of the digester modules 72 of the other three digesters 10 are then connected to the second pump outlet 48 of the second pump 38 using the second tank supply line 52. The withdrawal outlet 110 of the digester modules 72 of three digesters 10 are then connected to the first pump inlet 54 of the first pump 36 using the first tank withdrawal line 58. Similarly, the withdrawal outlet 110 of the digester modules 72 of the other three digesters 10 are then connected to the second pump inlet 56 of the second pump 38 using the second tank withdrawal line 60. The water inlets and outlets 86, 88 of each digester module 72 are connected to the hot water tank 18 using the water supply and return lines 90, 92. The gas outlets 122 of each digester module are connected to the gas storage tank 26 via the main gas line 124. The chute 14 is then connected to the main pump inlet 42 of the pump assembly 12 using the chute pipe 34. The pit dug for the tanks can then be filled in with the flanges 74 of the modules 72 exposed. The boiler 22 is then connected to the hot water tank 18, gas storage tank 26 and gas cylinder 24 and the thermal solar panel 20 can be connected to the hot water tank 18. The above installation method reduces the amount of time spend on site because the digester modules 72 contain all of the essential components that would otherwise need to be installed into each tank separately. The digester modules 72 can be simply and quickly loaded into each digester tank 11. This reduces the overall cost of the installation when compared to an installation in which the slurry supply and withdrawal pipes, heater coils and gas withdrawal pipe have to be installed into the tank in situ. It is also not necessary for an installer to work within the confined space of a digester tank chamber 62 as the digester module 72 can be installed into the tank from the outside.

In use, slurry, or any other mixture capable of undergoing anaerobic digestion, is fed into the chute 14 either directly or via a pre-digestion hydrolysis tank (not shown). The first and second chopper pumps 36, 38 of the pump assembly 12 draw the slurry through the chute pipe 34 and chop any solid material. The first and second pumps 36, 38 feed the slurry to the six digesters 10 through the first and second tank supply lines 50, 52 and the slurry is introduced into each tank 11 through the module supply pipe 94 of the digester module 72. The outlets 102, 104 of the supply pipe 94 spray the slurry into the tank chamber 62 which is filled to approximately 90% of its volume. The first and second pumps 36, 38 also act to withdraw slurry already in the tank through the module withdrawal pipe 96 and the first and second tank withdrawal lines 58, 60. Slurry is withdrawn through the withdrawal inlet 108 after the slurry has passed through the second mesh 80 of the module 72. The mesh 80 prevents any large pieces of matter blocking the withdrawal inlet 108. The slurry that is withdrawn from the tank is re-circulated through the first and second pumps 36, 38 and is fed back into the digesters 10 through the module supply pipe 94. This results in a continuous supply and withdrawal of slurry from the digesters 10 whilst maintaining the tank chamber 62 filled at approximately 90%.

Whilst slurry is continuously re-circulated within the digesters 10, hot water is continuously supplied to the heater coil 84 of each module 72 through the water inlet 86 and cooler water is continuously withdrawn from the heater coil 84 of the module 72 through the water outlet 88. The temperature of the water supplied is approximately 35° C. The water is supplied and returned to the hot water tank 18. The water can be heated by a number of methods including by the thermal solar panel 20 and/or the boiler 22. During initial start-up, the boiler 22 may have to be powered by a propane gas cylinder 24. However, after the plant 10 has operated for a period of time the boiler can be powered by the biogas stored in the gas storage tank 26.

The heater coil 84 of each digester module 72 heats the slurry within the digester tank chamber 62. The heating of the slurry results in anaerobic fermentation (or digestion) which generates methane (biogas). The methane generated is continuously drawn off the digester 10 using the gas withdrawal pipe 118 of the module 72. The biogas is then fed to the gas storage tank 26. As discussed above, the biogas may be used to power an electric generator and/or a boiler to generate hot water. Generated electricity may be supplied to buildings on-site and/or may be fed back into the national grid in order to generate an income. Any heat that the generator produces can be used to heat water for the hot water tank 18.

The digester plant 1 may be run for any appropriate time period, for example up to around 50 days and is preferably run for at least around 8 to 10 days. It is particularly preferred that it is run for around 10 to 20 days, although a time period of 25 to 50 days may be used. The first and second pumps 36, 38 of the pump assembly 12 can be used to drain slurry from the digesters 10. The slurry that has been anaerobically digested by the digester plant 1 is known as digestate and can be used as fertiliser. The digestate can be discharged to a slurry lagoon or can be discharged to a truck. The longer the plant 1 is run for, the better quality the fertiliser.

If one, or a number, of digesters 10 malfunction then the digester modules 72 can be easily removed from the tanks 11 without having to excavate the tanks. The digester modules 72 can then either be inspected on-site, without having to climb inside the digester tanks 11, or the modules 72 can be taken off-site and inspected in a workshop. If the modules 72 are faulty then they can either be replaced or repaired.

In this embodiment, once the digester modules 72 have been removed from the digester tanks 11 then there is nothing left in the tanks 11 that can be the source of a malfunction. This is because the pumps are located in a separate pump assembly 12 and any parts that can fail are part of the digester module 72. It is therefore easier, cheaper and quicker to identify any technical malfunctions.

Although in the foregoing description it has been described that the heater coil 84, module supply pipe 94, module withdrawal pipe 96 and gas withdrawal pipe 118 are part of a single removable digester module 72, in other embodiments each of the aforementioned parts may be part of two or more modules. Further, it may be desirable that only some of the aforementioned parts are part of a removable module and the remaining parts may be installed directly on or to the tank 11.

1. A digester for a digester plant, comprising:
a digester tank defining a digester tank chamber and having a module opening;
a digester module located in the module opening and extending into the digester tank chamber, wherein the digester module is removably attached to the digester tank and comprises a heater for heating material in the digester tank and at least one pipe having an inlet and an outlet, one of said inlet and outlet being inside the tank chamber and the other of said inlet and outlet being outside the tank chamber.

2. A digester according to claim 1, wherein said at least one pipe is selected from the group consisting of:
a supply pipe for supplying material to the digester tank, the supply pipe inlet being outside the tank chamber and the supply pip outlet being inside the tank chamber;
a withdrawal pipe for withdrawing material from the digester tank, the withdrawal pipe inlet being inside the tank chamber and the withdrawal pipe outlet being outside the tank chamber; and
a gas withdrawal pipe for withdrawing gas from the digester tank, the gas withdrawal pipe inlet being inside the tank chamber and the gas withdrawal pipe outlet being outside the tank chamber.

3. A digester according to claim 1 or 2, wherein the digester is or is configured to operate as an anaerobic digester.

4. A digester according to any preceding claim, wherein the digester tank is sealable so as to prevent the egress of air into the tank during use.

5. A digester according to any preceding claim, wherein the digester module comprises at least two of the supply pipe, the withdrawal pipe and the gas withdrawal pipe.

6. A digester according to any preceding claim, wherein the digester module comprises the supply pipe, the withdrawal pipe and the gas withdrawal pipe.

7. A digester according to any preceding claim, wherein the digester tank comprises two or more module openings, each module opening having a dedicated digester module located therein.

8. A digester according to any preceding claim, wherein the outlet of the supply pipe is towards the top of the tank chamber.

9. A digester according to any preceding claim, wherein the supply pipe comprises two outlets located inside the tank chamber.

10. A digester according to any preceding claim, wherein the withdrawal pipe extends towards the bottom of the tank chamber and the inlet is towards the bottom of the tank chamber.

11. A digester according to any preceding claim, wherein the inlet of the gas withdrawal pipe is towards the top of the tank chamber.

12. A digester according to any preceding claim, wherein heater comprises a length of tube having a water inlet a water outlet outside the tank chamber.

13. A digester according to claim 12, wherein the length of tube is coiled.

14. A digester according any preceding claim, wherein the digester tank comprises a first flange and the digester module comprises a second flange, wherein the second flange is detachably attached to the first flange.

15. A digester according to claim 14, wherein the supply pipe inlet and/or the withdrawal pipe outlet and/or the gas withdrawal pipe outlet is attached to the flange.

16. A digester according to any preceding claim, wherein the digester module comprises a cage for protecting the supply pipe and/or the withdrawal pipe and/or the gas withdrawal pipe.

17. A digester according to claim 16, wherein the cage comprises a mesh.

18. A digester according to any preceding claim, wherein the supply pipe is operatively connected to a material dosing tank.

19. A digester according to any preceding claim, wherein the supply pipe is operatively connected to a material hydrolysing tank.

20. A digester according to any preceding claim, wherein the gas withdrawal pipe is operatively connected to a gas storage vessel.

21. A module for use with the digester of any preceding claim.

22. A digester plant comprising at least one digester in accordance with any of claims 1 to 20.

23. A digester plant according to claim 22 comprising a plurality of digesters, one, more than one, or all of said digesters being in accordance with any one of claims 1 to 20.

24. A digester plant according to claim 23, wherein two or more of the plurality of digesters are connected to a controller or arranged to operate simultaneously, sequentially or with a lag between digestion starting in one digester and then being started in another digester.

25. A digester plant according to any one of claims 22 to 24, wherein the digester plant consists of 2 to 10 digesters.

26. A digester plant according to any one of claims 22 to 25, wherein the digester plant comprises a feedstock hydrolysis tank that is operatively connected to the or each digester.

27. A digester plant according to claim 26, wherein a filter or separator is provided in between the hydrolysis tank and the digester(s) to remove solids and any other undesirable substances from the hydrolysed feedstock before it is passed to the digester(s).

28. A digester plant according to any one of claims 22 to 27, wherein the digester plant comprises a feedstock dosing tank to temporarily store and then controllably feed the feedstock to the digester(s).

29. A digester plant according to any one of claims 22 to 28, wherein the digester plant comprises at least one biogas store operatively connected to the digester(s) to receive and store biogas.

30. A digester plant according to any one of claims 22 to 29, wherein the digester plant comprises at least one generator operatively connected to the digester(s).

31. A digester plant according to claim 30, wherein the generator is at least a 25 kWh generator set or larger.

32. A digester plant according to claim 30, wherein the generator is a 25 to 150 kWh generator set.

33. A method for digesting a biodegradeable material using a digester plant comprising one or more digesters, at least one of said digesters comprising:
a digester tank defining a digester tank chamber and having a module opening;
a digester module located in the module opening and extending into the digester tank chamber, wherein the digester module is removably attached to the digester tank and comprises a heater for heating biodegradeable material in the digester tank and at least one pipe having an inlet and an outlet, one of said inlet and outlet being inside the tank chamber and the other of said inlet and outlet being outside the tank chamber,
wherein the method comprises:
providing biodegradeable material in the digester tank chamber;
exposing the biodegradeable material in the digester tank chamber to bacteria capable of digesting the biodegradeable material to produce biogas;
operating the heater to heat the biodegradeable material; and
removing biogas produced by digestion of the biodegradeable material by the bacteria.

34. A method according to claim 33, wherein the digester plant comprises a plurality of digesters and the method involves carrying out the method set out above within two or more of said plurality of digesters simultaneously, sequentially or with a lag between the time at which biodegradeable material in each digester tank is exposed to the bacteria.

35. A method according to claim 34, wherein the method is effected under anaerobic conditions such that the method is an anaerobic digestion process.

36. A method according to claim any one of claims 33 to 35, wherein the method is carried out under conditions to produce biogas at a substantially constant flow rate.

37. A method according to any one of claims 33 to 36, wherein the method is effected in the or each digester to produce biogas at a rate of at least around 2 m³ per hour.

38. A method according to any one of claims 33 to 36, wherein the method is effected in the or each digester to produce biogas at a rate of around 2 to 10 m³ per hour.

39. A method according to any one of claims 33 to 38, wherein the method is carried out under mesophilic conditions.

40. A method according to any one of claims 33 to 39, wherein the biodegradeable material is exposed to bacteria in the digester tank chamber for up to around 50 days.

41. A method according to any one of claims 33 to 40, wherein the biodegradeable material is exposed to bacteria in the digester tank chamber for a time period of at least around 8 to 10 days.

42. A method according to any one of claims 33 to 39, wherein the biodegradeable material is exposed to bacteria in the digester tank chamber for around 10 to 20 days.

43. A method according to any one of claims 33 to 42, wherein the method comprises hydrolysis of the biodegradeable material before it is provided in the digester.

44. A method according to claim 43, wherein hydrolysis is effected at around 30 to 80° C.

45. A method according to claim 43 or 44, wherein hydrolysis is effected for up to around 7 days.

46. A method according to any one of claims 33 to 45, wherein the method further comprises filtering the hydrolysed biodegradeable material before providing it in the digester tank chamber.

47. A method according to any one of claims 33 to 46, wherein the method further comprises providing the biodegradeable material in a dosing tank from which can be controllably provided to the digester tank chamber.

48. A method according to any one of claims 33 to 47, wherein the method further comprises feeding the biogas removed from the digester tank chamber to a biogas store.

49. A method according to claims 33 to 48, wherein the biogas removed from the digester tank chamber is fed to a generator.

50. A method according to claim 49, wherein the generator is operated to generate heat and a portion of the generated heat is returned to the digester tank chamber to heat further biodegradeable material in the digester tank chamber.

51. A method according to claim 50, wherein the portion of heat returned may be at least 10 to 20%.
